(19) United States
(12) Patent Application Publication
(10) Pub. No.: US 2026/0166336 A1
(43) Pub. Date: Jun. 18, 2026

(54) OPTIMIZED RADIATION TREATMENT PLAN COMPLEXITY VARIATIONS QUANTIFICATION METHOD AND APPARATUS

(71) Applicant: Siemens Healthineers International AG, Steinhausen (CH)

(72) Inventors: Kellee Donnelly, Helsinki (FI); Emmi Ruokokoski, Helsinki (FI); Lauri Halko, Helsinki (FI)

(21) Appl. No.: 18/982,805

(22) Filed: Dec. 16, 2024

(51) Int. Cl. A61N 5/10

(52) U.S. Cl. CPC A61N 5/1031; A61N 5/1045; A61N 5/1048; A61N 2005/1074

(57) ABSTRACT

A control circuit can optimize a radiation treatment plan for a particular patient using a particular radiation treatment apparatus having at least one dynamic radiation delivery feature to provide an optimized radiation treatment plan, and then present to a user, via a user interface, a quantification of complexity variations that correspond to the optimized radiation treatment plan for the at least one dynamic radiation delivery feature.

### TECHNICAL FIELD

These teachings relate generally to treating a patient's planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-apparatus parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

### BRIEF DESCRIPTION OF DRAWINGS

The above needs are at least partially met through provision of the optimized radiation treatment plan complexity variations quantification method and apparatus described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a schematic visualization as configured in accordance with various embodiments of these teachings;

FIG. 4 comprises a screen shot as configured in accordance with various embodiments of these teachings;

FIG. 5 comprises a screen shot as configured in accordance with various embodiments of these teachings;

FIG. 6 comprises a screen shot as configured in accordance with various embodiments of these teachings;

FIG. 7 comprises a screen shot as configured in accordance with various embodiments of these teachings; and

FIG. 8 comprises charts as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Modulated arc treatments in radiotherapy, such as volumetric modulated arc therapy (VMAT), offer treatments that include modulating a combination of different machine axes, sometimes dynamically during the treatment. For example, in arc treatments today, the gantry, patient support surface, dose rate, multi-leaf collimator (MLC) leaves, and possibly the collimator itself are dynamic and therefore the beam is modulated with those axes. As linear accelerators and external beam radiotherapy treatment planning continue to evolve, it is foreseeable that even more axes will be dynamic during beam delivery and the trajectory describing the different axes as a function of time becomes increasingly complex.

Unfortunately, the applicant has determined that these increasing degrees of freedom and emerging modulation possibilities make quality assurance pertaining to a given optimized radiation treatment plan more difficult for a human to comprehend and apply. For example, prior efforts suggest capturing some aspects of the foregoing modulation possibilities by plotting the different machine axes from a radiation treatment plan using an X-Y charting paradigm, but the relation of the charted modulation feature to patient anatomy and beam geometry are not evident using such an approach. This informational disconnect can make it difficult or impossible for a human to intuit or otherwise grasp why plan quality may benefit from different modulation(s) in certain parts of the treatment arc.

Generally speaking, these various embodiments address the foregoing deficiencies. By one approach, these teachings provide for a control circuit optimizing a radiation treatment plan for a particular patient using a particular radiation treatment apparatus having at least one dynamic radiation delivery feature to provide an optimized radiation treatment plan. The control circuit can then present to a user, via a user interface, a quantification of complexity variations that correspond to the optimized radiation treatment plan for the at least one dynamic radiation delivery feature.

By one approach, the aforementioned particular radiation treatment apparatus has a plurality of different dynamic radiation delivery features. In such a case, presenting to the user the quantification of complexity variations can comprise presenting to the user a quantification of complexity variations that correspond to the optimized radiation treatment plan for at least one of the plurality of different dynamic radiation delivery features. By another approach, two or more different ones of the plurality of different dynamic radiation delivery features can be accounted for in the foregoing manner.

By way of example, the foregoing plurality of different dynamic radiation delivery features can include one or more of collimator jaw aperture complexity, multi-leaf collimator aperture complexity, multi-leaf collimator individual leaf movement, multi-leaf collimator rotation, a relative amount of radiation dosing at different control points, a physical speed of moving a radiation source, in-treatment radiation source movement, in-treatment controlled movement of the particular patient, treatment time, and/or patient support surface movement.

By one approach, these teachings will accommodate simultaneously presenting to the user, via the user interface, the aforementioned quantification of complexity variations that correspond to the optimized radiation treatment plan for at least two different ones of the plurality of different dynamic radiation delivery features. This simultaneous presentation, by one approach, can comprise simultaneously presenting to the user, via the user interface and via a shared presentation context (such as, as one illustrative example, a spider chart), the quantification of complexity variations that correspond to the optimized radiation treatment plan for at least two different ones of the plurality of different dynamic radiation delivery features. By one approach, the latter may comprise at least three different ones of the plurality of different dynamic radiation delivery features.

By one approach, these teachings can further accommodate conducting quality assurance for the optimized radiation treatment plan as a function of the presentation of the quantification of complexity variations. Presuming sufficient quality assurance, these teachings can then accommodate administering therapeutic radiation to the particular patient using the particular radiation treatment apparatus as a function of the optimized radiation treatment plan.

So configured, these teachings can enhance a user's interpretation of primary modulation factors through visualizations (including two-dimensional and/or three-dimensional views) with respect to the beam and patient anatomy. That enhanced interpretation, in turn, can help to reduce the duration of a quality assurance process while also helping to ensure a higher quality plan and therapeutic result for the patient.

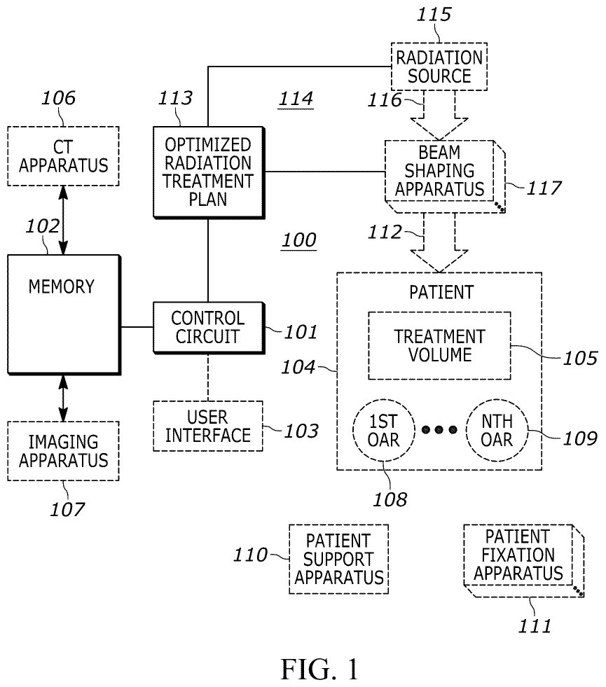

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware apparatus (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware apparatus (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be appreciated that the control circuit 101 may comprise a single integrated apparatus or may comprise a plurality of such circuits that work in cooperation with one another.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101). As with the control circuit 101, the memory 102 may comprise a singular structure or may comprise a plurality of memory apparatuses that collectively comprise the “memory” of this apparatus 100.

In addition to information such as optimization information for a particular patient and information regarding a particular radiation treatment apparatus as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

In this illustrative example the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless apparatuses, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-apparatus parameters during each of a plurality of sequential exposure fields. In this case the energy-based treatment plan is generated through an optimization process. For example, many such optimization processes are based upon use of so-called cost constraints. Cost constraints, in the context of iterative optimization approaches, refer to a predefined limitation that regulates the extent of one or more resources that can be allocated towards achieving the desired optimization objectives. Such constraints are factored into the optimization process to ensure that the solution adheres to boundaries set by such constraints. Accordingly, while the optimization process iteratively searches for an optimal solution by adjusting variables and assessing outcomes, the process simultaneously ensures that the total cost incurred in reaching that solution does not exceed the set cost constraint, thereby balancing the twin goals of optimization and management of the concern represented by the cost constraint.

As these teachings are not overly sensitive to any particular selection as regards optimization techniques, further elaboration in these regards is not provided here for the sake of brevity.

By one approach the control circuit 101 can operably couple to an energy-based treatment apparatus 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment apparatus/apparatuses. In a typical application setting the energy-based treatment apparatus 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment apparatus 114 may also include one or more patient support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

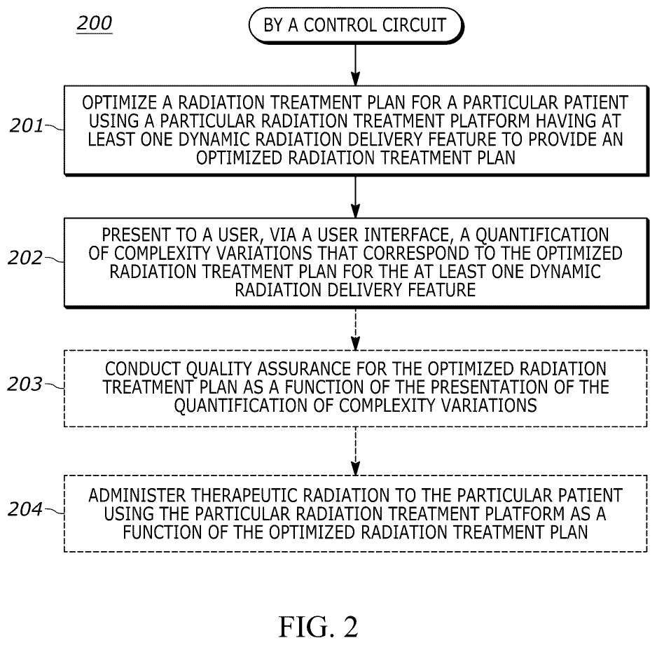

Referring now to FIG. 2, a process 200 that can be carried out, for example, in conjunction with the above-described application setting (and more particularly via the aforementioned control circuit 101) will be described. Generally speaking, this process 200 serves to facilitate generating an optimized radiation treatment plan 113 to facilitate treating a particular patient with therapeutic radiation using a particular radiation treatment apparatus per that optimized radiation treatment plan and then presenting a user with information regarding a quantification of complexity variations that correspond to that plan.

At block 201, this process 200 provides for the control circuit 101 optimizing a radiation treatment plan for a particular patient 104 using a particular radiation treatment apparatus 114 having at least one dynamic radiation delivery feature to provide an optimized radiation treatment plan 113. In a typical application setting, the particular radiation treatment apparatus 114 will have a plurality of different dynamic radiation delivery features. A non-exhaustive listing of at least some examples of different dynamic radiation delivery features include:
collimator jaw aperture complexity (where in the context of beam-shaping apparatuses, complexity will be understood to refer to such things as the shape of a formed aperture (including how smooth or jagged the sides of the aperture may be), the extent of leaf movement that is required to form a given shape, the speed of leaf movement that is required to form a given shape, collimator angles, and so forth;
multi-leaf collimator aperture complexity;
multi-leaf collimator individual leaf movement;
multi-leaf collimator rotation;
a relative amount of radiation dosing at different control points;
a physical speed of moving a radiation source;
in-treatment radiation source movement;
in-treatment controlled movement of the particular patient;
treatment time (where, for example, treatment time can refer, at a control point level, to the time it takes to deliver the control point; treatment time can even be shown for each axis (for example, referring to the time it takes for multi-leaf collimator leaves to move, for monitor units to be delivered, for the gantry to move, for the collimator to rotate between control points, and so forth); and/or
patient support surface movement (referring, for example, to couch rotation, speed, height adjustment, or other physical movements).

At block 202, the control circuit 101 then presents to a user, via a user interface 103, a quantification of complexity variations that correspond to the optimized radiation treatment plan 113 for the at least one dynamic radiation delivery feature. In the case where the complexity variations are for two or more different ones of the plurality of different dynamic radiation delivery features, the latter can comprise presenting this information via a shared presentation context for at least two different ones of the plurality of different dynamic radiation delivery features. Generally speaking, a shared presentation context will be understood to refer to a presentation axis, graph, chart, or the like, such that the information for multiple variables is presented via a common such context.

One non-limiting but useful example of a shared presentation context is a spider chart. A spider chart, also known as a radar chart or web chart, is a two-dimensional graphical tool used to display multivariate data. It consists of a series of axes, which radiate out from a central point like spokes on a wheel, with each axis representing one of the multiple variables. Data points are plotted along these axes and are typically connected by a line, or often a filled area, forming a shape that can be interpreted by a human viewer to understand the relative strengths and weaknesses of the subject under analysis across different dimensions.

Accordingly, when presenting information for, say, three or four different dynamic radiation delivery complexity features (such as a multi-leaf collimator-based complexity feature, a control point-based dosing-based complexity feature; a radiation source movement-based complexity feature, and a fluence-based complexity feature), each of the aforementioned spider chart axes may correspond to a single such complexity feature.

As another illustrative example, for treatments with static beams or arcs combined with static beams or arcs with dense control points, these teachings would accommodate plotting complexity variations as a function of a control point index instead of angle to possibly better present variations inside the static beams/denser control points as well.

Further illustrative examples in the foregoing regards are provided further below.

At optional block 203, this process 200 can provide for conducting quality assurance for the optimized radiation treatment plan as a function of the presentation of the quantification of complexity variations. Quality assurance (also referred to by the acronym QA) in the context of radiation therapy is a systematic process of checking to ensure that the treatment being provided meets established standards of quality. This can include verifying that the treatment plan is accurately calculated and that the safety of the patient is maintained throughout the treatment process. The goal of QA is to ensure that the treatment is both safe and effective.

Quality assurance is useful for assessing an optimized radiation treatment plan for at least the following reasons.

Radiation therapy involves the delivery of high-energy radiation to a targeted area, usually a tumor, with the intent to destroy cancer cells. To minimize damage to surrounding healthy tissue, the radiation dose must be delivered accurately according to the treatment plan. QA procedures can check that the radiation dose calculated in the plan is the same as the dose to be delivered.

QA can be important to patient safety. In particular, QA can help to prevent overexposure to radiation (which can lead to serious side effects) and underexposure (which can result in ineffective treatment).

In radiation therapy, treatments are often given over several sessions. QA can help to ensure that each treatment session is consistent with the treatment plan. This includes checks on patient positioning, imaging, and treatment delivery.

QA can also provide a documented trail of checks and calibrations that can be reviewed in the case of any discrepancies or incidents. The presentation of information described herein can play a simple yet effective role in these regards.

Generally speaking, the presentation of a quantification of complexity variations for each of a plurality of dynamic radiation delivery features as described herein can greatly simplify, or even make possible, review and consideration of such factors by a human observer.

Optional block 204, this process 200 will also accommodate administering therapeutic radiation to the particular patient 100 for using the particular radiation treatment apparatus 114 as a function of the optimized radiation treatment plan 113, particularly after that optimized radiation treatment plan 113 has passed quality assurance as informed by the aforementioned presentation of the quantification of complexity variations.

Further examples that comport with these teachings will now be presented. It will be understood that the specific details of these examples are intended to serve an illustrative purpose and are not intended to suggest any particular limitations with respect to these teachings.

The following examples make use of the following modulations, it being understood that additional modulations and/or substitute modulations can certainly be utilized: aperture shape complexity (such as, for example, a modulation factor (MF) or selected complexity index (CI) per control point or degree of arc); a proportion of irradiation (for example, a proportion (%) of dosimetric contribution per control point); and a speed of delivery/gantry speed (such as, for example, gantry speed expressed in seconds per degree of arc, seconds per control point, or even revolutions per minute) (especially useful when the dose rate or leaf movement cause the gantry to slow down to provide time to accommodate such movements).

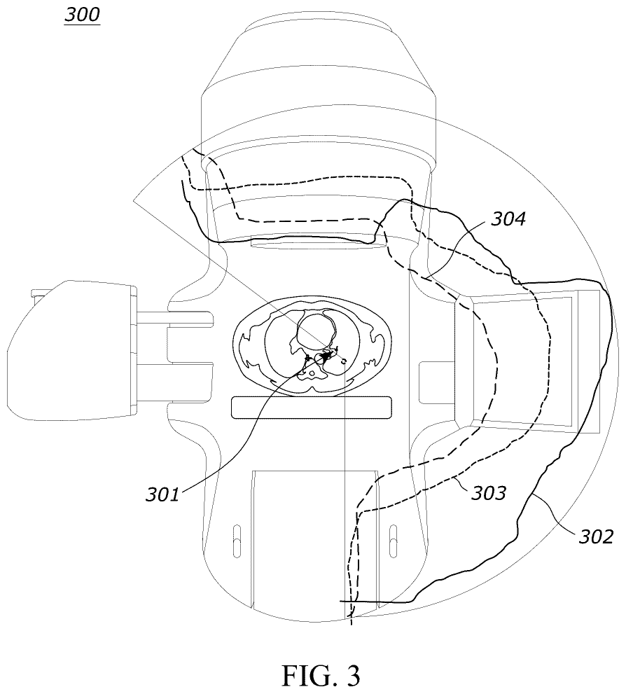

FIG. 3 presents a schematic visualization 300 that is shown with respect to the isocenter position 301 (or other useful patient case representation). Three lines are shown in this visualization 300 that indicate values for each of an aperture shape complexity index 302, a dose proportion 303, and gantry speed 304 as the source of radiation moves arcuately via the gantry around the isocenter 301.

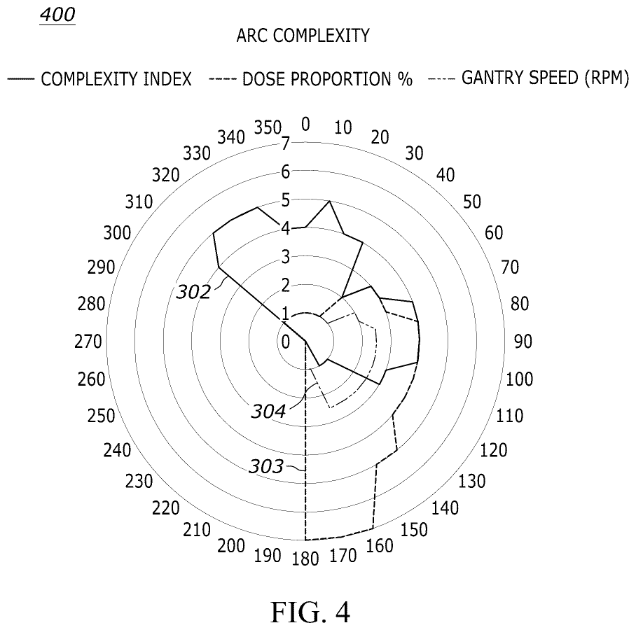

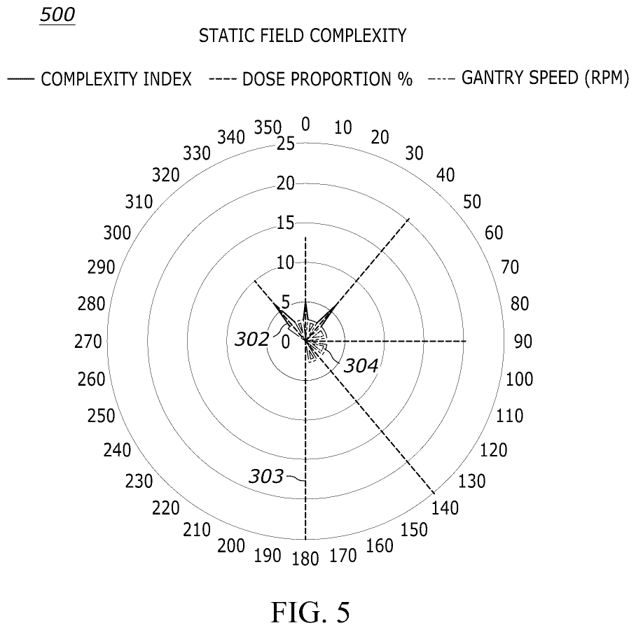

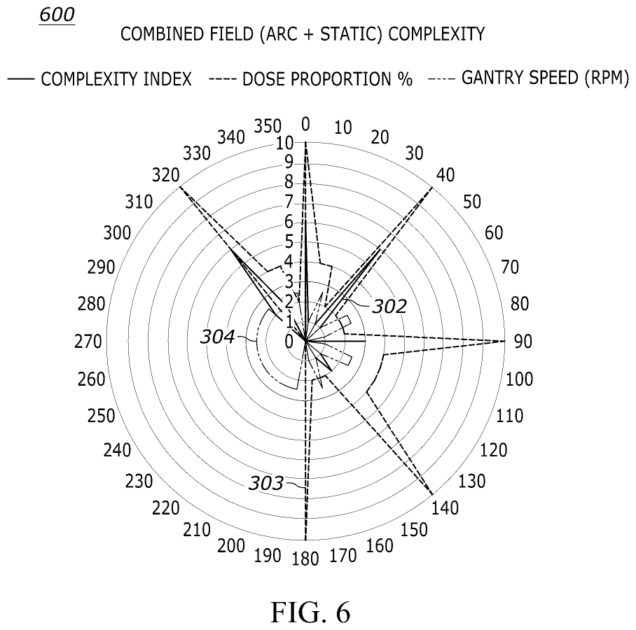

FIG. 4 then presents a corresponding spider chart 400 that depicts a quantification of the foregoing three complexity variations that correspond to arc complexity for an illustrative optimized volumetric modulated arc therapy radiation treatment plan. FIG. 5 presents a corresponding spider chart 500 that depicts a quantification of the foregoing three complexity variations that correspond to static field complexity for an illustrative optimized intensity-modulated radiation therapy radiation treatment plan. And FIG. 6 presents a corresponding spider chart 600 that depicts a quantification of the foregoing three complexity variations that correspond to a combined field complexity of both arc complexity and static field complexity for a hybrid approach involving both arc and static portions.

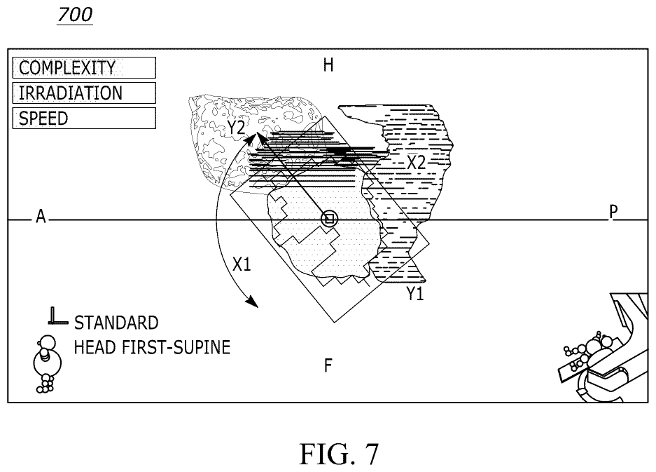

As should already be evident, these teachings are highly flexible in practice and will accommodate any of a variety of modifications and/or supplemental features. FIG. 7 presents one illustrative example in these regards. In this figure, a screen shot 700 presents a two-dimensional representation of the same data as presumed above. In this example, this data is shown from a so-called beam's eye view (per control point or arc degree). Such a presentation may be useful in some application settings to help a user conduct a possibly more thorough analysis.

So configured, these teachings can provide a user with more information than is typically available today while simultaneously providing that information in a form that is relatively easy to understand and cognitively apply. The quantification of complexity variations provided herein can also help to avoid possible confusion with regards to monitor unit/degree information. It will be appreciated that the visualization and quantification of a complexity metric per control point with respect to an arc can help a user to understand and separate aperture complexity from dose contributions. And it will be further appreciated that the described visualization and quantification of gantry speed can help the user relate the impact of both dose contribution and/or modulation in certain parts of the arc.

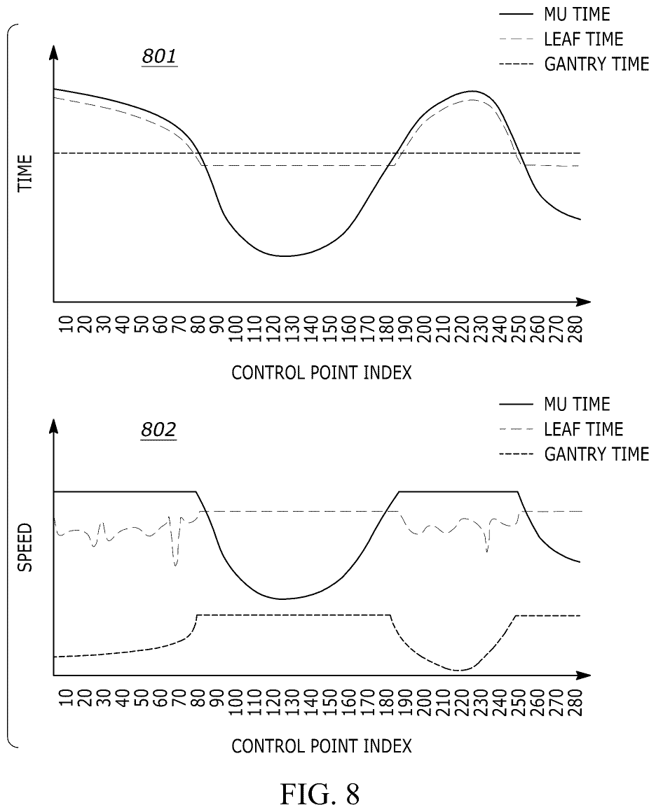

And so configured, these teachings offer radiation treatment planners unique opportunities to present or explain the ramifications of a given plan. As one example, such quantifications can help to spotlight where larger dose contribution or higher complexity is required, and that, in turn, can lead to trying to improve the treatment plan. This may comprise, for example, adding more control points to a static beam or arc, or by adding an extra beam or arc with possibly different collimator angle or rotating collimator settings. FIG. 8 presents an illustrative example in these regards with charted examples of delivery time and speed for a selection of machine axes as a function of a control point index (801 and 802 respectively).

As another example, such quantification information can be used with patients when explaining the nature of the beam delivery that they may experience. A treatment staff member might, for example, present the described quantification of complexity variations to a patient while saying something like, “You may notice that the beam rotates very fast on your right hand side, but then slows down and stops as it rotates above you. This is normal and expected as part of your planned treatment.”

Further aspects of these teachings are provided by the subject matter of the following clauses (where it will be understood that any of these clauses can be combined with any one of more of the other clauses as appropriate).

Clause 1. A method comprising: by a control circuit: optimizing a radiation treatment plan for a particular patient using a particular radiation treatment apparatus having at least one dynamic radiation delivery feature to provide an optimized radiation treatment plan; presenting to a user, via a user interface, a quantification of complexity variations that correspond to the optimized radiation treatment plan for the at least one dynamic radiation delivery feature.

Clause 2. The method of clause 1 wherein the particular radiation treatment apparatus has a plurality of different dynamic radiation delivery features and wherein presenting to the user the quantification of complexity variations comprises presenting to the user, via the user interface, a quantification of complexity variations that correspond to the optimized radiation treatment plan for at least one of the plurality of different dynamic radiation delivery features.

Clause 3. The method of clause 2 wherein presenting to the user, via the user interface, the quantification of complexity variations that correspond to the optimized radiation treatment plan for at least one of the plurality of different dynamic radiation delivery features comprises presenting to the user, via the user interface, a quantification of complexity variations that correspond to the optimized radiation treatment plan for at least two different ones of the plurality of different dynamic radiation delivery features.

Clause 4. The method of clause 3 wherein presenting to the user, via the user interface, a quantification of complexity variations that correspond to the optimized radiation treatment plan for at least two different ones of the plurality of different dynamic radiation delivery features comprises simultaneously presenting to the user, via the user interface, the quantification of complexity variations that correspond to the optimized radiation treatment plan for at least two different ones of the plurality of different dynamic radiation delivery features.

Clause 5. The method of clause 4 wherein simultaneously presenting to the user, via the user interface, the quantification of complexity variations that correspond to the optimized radiation treatment plan for at least two different ones of the plurality of different dynamic radiation delivery features comprises simultaneously presenting to the user, via the user interface and via a shared presentation context, the quantification of complexity variations that correspond to the optimized radiation treatment plan for at least two different ones of the plurality of different dynamic radiation delivery features.

Clause 6. The method of clause 5 wherein the shared presentation context comprises a spider chart.

Clause 7. The method of any of clause 2 through 6 wherein the plurality of different dynamic radiation delivery features include at least some of: collimator jaw aperture complexity; multi-leaf collimator aperture complexity; multi-leaf collimator individual leaf movement; multi-leaf collimator rotation; relative amount of radiation dosing at different control points; physical speed of moving a radiation source; in-treatment radiation source movement; in-treatment controlled movement of the particular patient; treatment time; patient support surface movement.

Clause 8. The method of clause 7 wherein presenting to the user, via the user interface, the quantification of complexity variations that correspond to the optimized radiation treatment plan for at least one of the plurality of different dynamic radiation delivery features comprises presenting to the user, via the user interface, a quantification of complexity variations that correspond to the optimized radiation treatment plan for at least three different ones of the plurality of different dynamic radiation delivery features, wherein the at least three different dynamic radiation delivery complexity features are selected from: a multi-leaf collimator-based complexity feature; a control point-based dosing-based complexity feature; a radiation source movement-based complexity feature; a fluence-based complexity feature.

Clause 9. The method of any of clause 1 through 8 further comprising: conducting quality assurance for the optimized radiation treatment plan as a function of the presentation of the quantification of complexity variations.

Clause 10. The method of clause 1 through 9 further comprising:
administering therapeutic radiation to the particular patient using the particular radiation treatment apparatus as a function of the optimized radiation treatment plan.

Clause 11. An apparatus comprising: a control circuit configured to: optimize a radiation treatment plan for a particular patient using a particular radiation treatment apparatus having at least one dynamic radiation delivery feature to provide an optimized radiation treatment plan; present to a user, via a user interface, a quantification of complexity variations that correspond to the optimized radiation treatment plan for the at least one dynamic radiation delivery feature.

Clause 12. The apparatus of clause 11 wherein the particular radiation treatment apparatus has a plurality of different dynamic radiation delivery features and wherein the control circuit is configured to present to the user the quantification of complexity variations by presenting to the user, via the user interface, a quantification of complexity variations that correspond to the optimized radiation treatment plan for at least one of the plurality of different dynamic radiation delivery features.

Clause 13. The apparatus of clause 12 wherein the control circuit is configured to present to the user, via the user interface, the quantification of complexity variations that correspond to the optimized radiation treatment plan for at least one of the plurality of different dynamic radiation delivery features by presenting to the user, via the user interface, a quantification of complexity variations that correspond to the optimized radiation treatment plan for at least two different ones of the plurality of different dynamic radiation delivery features.

Clause 14. The apparatus of clause 13 wherein the control circuit is configured to present to the user, via the user interface, a quantification of complexity variations that correspond to the optimized radiation treatment plan for at least two different ones of the plurality of different dynamic radiation delivery features by simultaneously presenting to the user, via the user interface, the quantification of complexity variations that correspond to the optimized radiation treatment plan for at least two different ones of the plurality of different dynamic radiation delivery features.

Clause 15. The apparatus of clause 14 wherein the control circuit is configured to simultaneously present to the user, via the user interface, the quantification of complexity variations that correspond to the optimized radiation treatment plan for at least two different ones of the plurality of different dynamic radiation delivery features by simultaneously presenting to the user, via the user interface and via a shared presentation context, the quantification of complexity variations that correspond to the optimized radiation treatment plan for at least two different ones of the plurality of different dynamic radiation delivery features.

Clause 16. The apparatus of clause 15 wherein the shared presentation context comprises a spider chart.

Clause 17. The apparatus of any of clauses 12 through 16 wherein the plurality of different dynamic radiation delivery features include at least some of: collimator jaw aperture complexity; multi-leaf collimator aperture complexity; multi-leaf collimator individual leaf movement; multi-leaf collimator rotation; relative amount of radiation dosing at different control points; physical speed of moving a radiation source; in-treatment radiation source movement; in-treatment controlled movement of the particular patient; treatment time; patient support surface movement

Clause 18. The apparatus of clause 17 wherein the control circuit is configured to present to the user, via the user interface, the quantification of complexity variations that correspond to the optimized radiation treatment plan for at least one of the plurality of different dynamic radiation delivery features by presenting to the user, via the user interface, a quantification of complexity variations that correspond to the optimized radiation treatment plan for at least three different ones of the plurality of different dynamic radiation delivery features, wherein the at least three different dynamic radiation delivery complexity features comprise: a multi-leaf collimator-based complexity feature; a control point-based dosing-based complexity feature; a radiation source movement-based complexity feature; a fluence-based complexity feature.

Clause 19. The apparatus of any of clauses 11 through 18 wherein the control circuit is further configured to: facilitate quality assurance for the optimized radiation treatment plan as a function of the presentation of the quantification of complexity variations.

Clause 20. The apparatus of any of clauses 11 through 19 wherein the control circuit is further configured to: administer therapeutic radiation to the particular patient using the particular radiation treatment apparatus as a function of the optimized radiation treatment plan.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A method comprising:
by a control circuit:
optimizing a radiation treatment plan for a particular patient using a particular radiation treatment apparatus having at least one dynamic radiation delivery feature to provide an optimized radiation treatment plan;
presenting to a user, via a user interface, a quantification of complexity variations that correspond to the optimized radiation treatment plan for the at least one dynamic radiation delivery feature.

2. The method of claim 1 wherein the particular radiation treatment apparatus has a plurality of different dynamic radiation delivery features and wherein presenting to the user the quantification of complexity variations comprises presenting to the user, via the user interface, a quantification of complexity variations that correspond to the optimized radiation treatment plan for at least one of the plurality of different dynamic radiation delivery features.

3. The method of claim 2 wherein presenting to the user, via the user interface, the quantification of complexity variations that correspond to the optimized radiation treatment plan for at least one of the plurality of different dynamic radiation delivery features comprises presenting to the user, via the user interface, a quantification of complexity variations that correspond to the optimized radiation treatment plan for at least two different ones of the plurality of different dynamic radiation delivery features.

4. The method of claim 3 wherein presenting to the user, via the user interface, a quantification of complexity variations that correspond to the optimized radiation treatment plan for at least two different ones of the plurality of different dynamic radiation delivery features comprises simultaneously presenting to the user, via the user interface, the quantification of complexity variations that correspond to the optimized radiation treatment plan for at least two different ones of the plurality of different dynamic radiation delivery features.

5. The method of claim 4 wherein simultaneously presenting to the user, via the user interface, the quantification of complexity variations that correspond to the optimized radiation treatment plan for at least two different ones of the plurality of different dynamic radiation delivery features comprises simultaneously presenting to the user, via the user interface and via a shared presentation context, the quantification of complexity variations that correspond to the optimized radiation treatment plan for at least two different ones of the plurality of different dynamic radiation delivery features.

6. The method of claim 5 wherein the shared presentation context comprises a spider chart.

7. The method of claim 2 wherein the plurality of different dynamic radiation delivery features include at least some of:
collimator jaw aperture complexity;
multi-leaf collimator aperture complexity;
multi-leaf collimator individual leaf movement;
multi-leaf collimator rotation;
relative amount of radiation dosing at different control points;
physical speed of moving a radiation source;
in-treatment radiation source movement;
in-treatment controlled movement of the particular patient;
treatment time;
patient support surface movement.

8. The method of claim 7 wherein presenting to the user, via the user interface, the quantification of complexity variations that correspond to the optimized radiation treatment plan for at least one of the plurality of different dynamic radiation delivery features comprises presenting to the user, via the user interface, a quantification of complexity variations that correspond to the optimized radiation treatment plan for at least three different ones of the plurality of different dynamic radiation delivery features, wherein the at least three different dynamic radiation delivery complexity features are selected from:
a multi-leaf collimator-based complexity feature;
a control point-based dosing-based complexity feature;
a radiation source movement-based complexity feature;
a fluence-based complexity feature.

9. The method of claim 1 further comprising:
conducting quality assurance for the optimized radiation treatment plan as a function of the presentation of the quantification of complexity variations.

10. The method of claim 1 further comprising:
administering therapeutic radiation to the particular patient using the particular radiation treatment apparatus as a function of the optimized radiation treatment plan.

11. An apparatus comprising:
a control circuit configured to:
optimize a radiation treatment plan for a particular patient using a particular radiation treatment apparatus having at least one dynamic radiation delivery feature to provide an optimized radiation treatment plan;
present to a user, via a user interface, a quantification of complexity variations that correspond to the optimized radiation treatment plan for the at least one dynamic radiation delivery feature.

12. The apparatus of claim 11 wherein the particular radiation treatment apparatus has a plurality of different dynamic radiation delivery features and wherein the control circuit is configured to present to the user the quantification of complexity variations by presenting to the user, via the user interface, a quantification of complexity variations that correspond to the optimized radiation treatment plan for at least one of the plurality of different dynamic radiation delivery features.

13. The apparatus of claim 12 wherein the control circuit is configured to present to the user, via the user interface, the quantification of complexity variations that correspond to the optimized radiation treatment plan for at least one of the plurality of different dynamic radiation delivery features by presenting to the user, via the user interface, a quantification of complexity variations that correspond to the optimized radiation treatment plan for at least two different ones of the plurality of different dynamic radiation delivery features.

14. The apparatus of claim 13 wherein the control circuit is configured to present to the user, via the user interface, a quantification of complexity variations that correspond to the optimized radiation treatment plan for at least two different ones of the plurality of different dynamic radiation delivery features by simultaneously presenting to the user, via the user interface, the quantification of complexity variations that correspond to the optimized radiation treatment plan for at least two different ones of the plurality of different dynamic radiation delivery features.

15. The apparatus of claim 14 wherein the control circuit is configured to simultaneously present to the user, via the user interface, the quantification of complexity variations that correspond to the optimized radiation treatment plan for at least two different ones of the plurality of different dynamic radiation delivery features by simultaneously presenting to the user, via the user interface and via a shared presentation context, the quantification of complexity variations that correspond to the optimized radiation treatment plan for at least two different ones of the plurality of different dynamic radiation delivery features.

16. The apparatus of claim 15 wherein the shared presentation context comprises a spider chart.

17. The apparatus of claim 12 wherein the plurality of different dynamic radiation delivery features include at least some of:
collimator jaw aperture complexity;
multi-leaf collimator aperture complexity;
multi-leaf collimator individual leaf movement;
multi-leaf collimator rotation;
relative amount of radiation dosing at different control points;
physical speed of moving a radiation source;
in-treatment radiation source movement;
in-treatment controlled movement of the particular patient;
treatment time;
patient support surface movement.

18. The apparatus of claim 17 wherein the control circuit is configured to present to the user, via the user interface, the quantification of complexity variations that correspond to the optimized radiation treatment plan for at least one of the plurality of different dynamic radiation delivery features by presenting to the user, via the user interface, a quantification of complexity variations that correspond to the optimized radiation treatment plan for at least three different ones of the plurality of different dynamic radiation delivery features, wherein the at least three different dynamic radiation delivery complexity features comprise:
a multi-leaf collimator-based complexity feature;
a control point-based dosing-based complexity feature;
a radiation source movement-based complexity feature;
a fluence-based complexity feature.

19. The apparatus of claim 11 wherein the control circuit is further configured to:
facilitate quality assurance for the optimized radiation treatment plan as a function of the presentation of the quantification of complexity variations.

20. The apparatus of claim 11 wherein the control circuit is further configured to:
administer therapeutic radiation to the particular patient using the particular radiation treatment apparatus as a function of the optimized radiation treatment plan.
